(19) United States
(12) Patent Application Publication
(10) Pub. No.: US 2025/0345138 A1
(43) Pub. Date: Nov. 13, 2025

(54) MEDICALIMAGINGCOMPATIBLERADIOLUCENTACTUATIONOFARTICULATING ROBOTICMUSCULATURE

(71) Applicant: Michael Campagna, Tampa, FL (US)

(72) Inventor: Michael Campagna, Tampa, FL (US)

(21) Appl. No.: 18/744,485

(22) Filed: Jun. 14, 2024

Related U.S. Application Data
(63) Continuation-in-part of application No. 18/660,233, filed on May 10, 2024.

(51) Int. Cl. A61B 34/37; A61B 34/30; B25J 9/00; B25J 9/10; F03G 7/00; H01B 1/04; H01B 1/24

(52) U.S. Cl. CPC A61B 34/37; B25J 9/0012; B25J 9/1075; F03G 7/0121; H01B 1/04; H01B 1/24; A61B 2034/305

(57) ABSTRACT

A robot joint control that eliminates backlash and utilizes Polymer (EAP) muscle.

### RELATED PATENTS

This application claims priority to and is a continuation-in-part of U.S. patent application Ser. No. 18/660,233, titled “MEDICAL IMAGING COMPATIBLE RADIOLUCENT ACTUATION,” filed on May 10, 2024, by Michael Campagna, and is incorporated by reference in its entirety.

### TECHNICAL FIELD

The present invention relates generally to robotics, and more particularly to radiolucent CT & MRI Compatible Articulation and Actuation of Surgical Robotics.

The applications U.S. Divisional patent application Ser. No. 18/388,856, filed on Nov. 12, 2023, claimed priority to U.S. patent application Ser. No. 16/908,620, filed on Jun. 22, 2020, which claims priority to U.S. Provisional Patent Application No. 63/038,743, titled “MEDICAL IMAGING COMPATIBLE RADIOLUCENT ACTUATION OF TRANSLATION ROTATION ARTICULATION CIRCUMDUCTION,” filed on Jun. 12, 2020, by Michael Campagna. The above applications are incorporated by reference only, and no claim to priority is made.

### BACKGROUND

The present state of the art in minimally-invasive surgical robotics is characterized by cable-actuated surgical robotic end effectors, which are primarily metallic in composition and utilize metallic compositions as the means of articulation and of electrical transmission of current and are thereby inaccessible to usage within radiographic and magnetic imaging bores and sensors due to the incidence of high density and high attenuation artifact as well as the MRI “Black Hole” artifact occurring do to metal usage in the surgical robotics end effectors. Furthermore, it is well known in the art that the cable-actuated surgical robotic end-effectors are subject to unexpected motion due to the coupled-motion phenomenon between the end-effector's pseudo wrist and grippers, whereby each said degree of freedom is no longer independent but is coupled via the actuating cable. This coupling phenomenon causes the end-effector to “Freeze Up.” This tendency in cable-actuated surgical robotics end-effectors is further exacerbated by the well-known tendency of the cable actuation to experience both “backlash” and hysteresis upon the cable, often causing unexpected backward-retrograde motion of the end-effector contrary to the issued commands of the surgeon during the performance of dissection and anastomosis. It is known in the art of cable-driven surgical robotic end effectors utilized in microsurgery that Backlash, Hysteresis, and the coupled motion phenomenon introduce unexpected motion and uncertainty into the usage of surgical end effectors and thereby present significant challenges to the precise and accurate control of the end-effector.

The above is a recognized problem that has existed within art for a long time with no solution. Furthermore, current surgical robotics control systems utilize cable actuation as the means of robotic end effector joint articulation. They are thereby fundamentally non-naturalistic and non-intuitive as this results in robotic joints that exaggerate as opposed to replicating the actual motions performed by the surgeon due to Backlash, Hysteresis, and coupled motion phenomenon. It is well known in the art, and the subject of much discussion within medical and robotics journals, that cable-actuated surgical robotic end-effectors are subject to unexpected motion due to the coupled-motion phenomenon between the end-effector's pseudo wrist and grippers, whereby each said degree of freedom is no longer independent but is coupled via the actuating cable. This tendency in cable-actuated surgical robotics end-effectors is further exacerbated by the well-known tendency of the cable actuation experiencing both backlash and hysteresis upon the cable, often causing unexpected backward-retrograde motion of the end-effector contrary to the issued commands of the surgeon during the performance of dissection and anastomosis.

This reliance on rotational wire/cable actuation also introduces a corresponding buildup of slack and or tension into the rotational wire/cable means of articulation, which must be perpetually “clutched” or reset in order to account for the buildup and release of wire/cable tension. This buildup and sudden abrupt and unexpected release of cable tension causes slippage and introduces unexpected backlash retrograde motion to the end-effector, which can cause unexpected motion and thereby seriously compromise safe and controlled usage during delicate microsurgery, thereby necessitating the “clutching” and resetting of the cable tension and slack within the rotational wire/cable means of robotic joint articulation. As such, the imprecise slippage and unexpected backlash, which introduces error into robotic microsurgery and thereby necessitates the continuous “resetting” and “clutching” of the wire/cable means of actuation, represents a real problem with the current surgical robotics.

Also, the current cable surgical robots are unable to remain within a live MRI/CT imaging bore and, therefore, unable to interface with the three-dimensional magnetic and radiographic, MRI & CT (3D) image-guided surgery and are only able to utilize low-resolution photo-acoustic 3D image guidance which primarily identifies blood vessels as opposed to soft tissues. An additional problem in the art is that current surgical robots are unable to function in an MRI & CT 3D image-guided surgery (IGS).

To summate, it is well known in the art of cable driven surgical robotic end effectors utilized in microsurgery that backlash, hysteresis, and coupled motion present significant challenges to the precise and accurate control of the end effector. When state-of-the-art actuated surgical robotics are subject to both uncontrolled and imprecise motion due to backlash and hysteresis during microsurgical procedures, are prone to freeze due to the coupling phenomenon during microsurgical procedures, and are thereby unable to fully deliver the precision control that usage during such microsurgical procedures demands, a real problem exists within the art. The issues identified have existed within the art for an extended time with no solution.

What is needed in art is an approach to overcoming the problems identified.

### SUMMARY

The present invention, “Micra-Arm” is an acronym for “Medically Imaging Compatible Radiolucent Actuation of Articulating Robotic Musculature,” which describes the function of the present invention and which translates as medically imaging compatible radiolucent actuation of articulating robotic musculature. The method and apparatus of Micra-Arm herein disclose a non-metallic means of providing predictable, stable, and controlled electrically powered medically imaging compatible, radiolucent actuation of articulating robotic musculature which functions with reliable and pinpoint precision within the CT and MRI Imaging Bore, without the accustomed backlash, hysteresis, slippage, sudden uncontrolled motion, or stalled inaction due to the coupling phenomenon and the commensurate need for reset and clutching which plagues the wire/cable actuation of known surgical robotics. Micra-Trac eliminates these cable actuation-related difficulties and presents stable, smooth, and precise electrical actuation of radiolucent surgical robotics and surgical robotics end effectors. The improvements are achieved via the utilization of the migratory action provided by coordinated antagonistic pairs of inverse proportional electroactive polymers (EAP) flexor extensor artificial muscles acting upon a substantially rhomboidal/lozenge-shaped effort arm of a rotatable radiolucent anatomical support member of the radiolucent rotatable joint. These EAP artificial muscles are delivered electrical power via radiolucent non-metallic flexible carbon-based conductive electrical transmission wire, which can function within the CT and MRI Imaging Bores, thereby providing rotation of the joint around the one central pivot point of the rotatable radiolucent joint.

In this manner, the present approach is enabled to replicate the natural motion of the human wrist and shoulder circumduction joints as well as the elbow's flexion/extension joints precisely, and forefinger/thumb pincer or forefinger/middle finger scissoring function, in a smooth, reliable and minutely controlled manner within the CT and MRI imaging bores, which at present is not possible with state of the art wire/cable actuated surgical robotics due to the cable actuation specific problems of backlash, hysteresis, and coupling errors. Furthermore, the present Micra-Arm approach enables the usage of radiolucent antagonistic inverse-proportional pairs of electroactive EAP flexor extensors within a radiolucent joint within the CT and MRI, where the surgical end-effector may work in concert with the CT and MRI 3D image guidance systems (IGS), which at present, prior art end-effectors cannot achieve. The current state-of-the-art metal-based end effectors cannot function within the CT and MRI imaging bores and are thereby enabled to work in concert with only photo-acoustic IGS. Furthermore, the present Micra-Arm approach enables minute and controlled precision of actuation and articulation of surgical robotics in microsurgery without the difficulties of wire/cable slippage and backlash, which require constant “clutching” and “resetting” that currently plague the current state of the art in robotic surgical end effectors.

The Micra-Arm approach, where electroactive polymer (EAP) artificial muscles are made to function and powered entirely radiolucent and non-metallic, enables them to function within the CT and MRI—configured as antagonistic pairs of flexor extensor artificial muscles in the form of non-metallic parallel plate, dielectric or ionic elastomer actuators, which convert electrical impulses into mechanical movement. EAP muscles have the ability to change in shape and size when stimulated by an electric field acting upon the carbon-based stretchable non-metallic dielectric parallel electrode plates, which transform the application of voltage into compression of the soft silicone or polyvinyl siloxane (PVS), also called polyvinyl siloxane, vinyl polysiloxane (VPS), or vinylpolysiloxane, PVS Elastomer sandwiched between these said parallel electrode plates, which said compression and release of compression may then be utilized to replicate the actions of organic flexor extensor and muscle fiber. This compressibility of the EAP is somewhat akin to how the incompressibility of liquids is utilized as the operative method in hydraulics, in that the elastomer is also incompressible and thereby responds to the decrease in the dimension of width or height via an instantaneous and corresponding increase in length.

The corollary is also true that the elastomer responds to a decrease in the dimension of length via an instantaneous and corresponding increase in the width or height dimensions. In this manner, when voltage is applied, the EAP muscle compresses into a thinner and flatter yet wider shape, maintaining the same volumetric value. Alternately, when voltage is no longer applied, the EAP muscle returns to its original thicker shape, which maintains the same volumetric value. Furthermore, this compressibility and return to the original thicker shape may be selectively altered volt by volt, thereby delivering the most precise and controlled artificial muscle reactions, which may then be employed within the radiolucent surgical end effector for delicate and controlled micro-surgery. To that end, the present invention utilizes non-metallic electroactive polymer EAP artificial muscles in concert with flexible non-metallic graphene impregnated cotton (or other fiber), or non-metallic flexible silicone-carbon nanotube electrical transmission array carbon electrical array as the means of medically imaging compatible radiolucent means of delivering electrical voltage to the EAP musculature. The present invention utilizes these radiolucent antagonistic inverse-proportional pairs of EAP flexor extensors within the micra-trac radiolucent joints to replicate the function of actual anatomical organic flexor extensor muscle pairs in a smooth and controlled progression, precisely, as the flexor of the pair of antagonistic flexor/extensors is made to increase in thickness via the coordinated decrease in application of voltage, the extensor of the antagonistic pair of flexor/extensors is made to inverse proportionally decrease in thickness via the coordinated increase in the application of current. Alternatively, as the flexor of the pair of antagonistic flexors/extensors is made to decrease in thickness via the coordinated increase in the application of voltage, the extensor of the antagonistic pair of flexor extensors inverse is made to proportionally increase in thickness via the coordinated decrease in the application of voltage. Thus, the present invention, via the precise application of micro control of voltage to the antagonistic pair of electroactive polymer flexor extensors, enables the inverse proportional pair(s) of antagonistic electroactive flexor extensors to be controlled with an absolute smooth and selective precision, thereby delivering a level of control to robotic microsurgery that both greatly exceeds and is unmatched by the current state of the art usage of wire/cable rotational actuation of the surgical end effector, without being prone to the pitfalls of backlash and clutching, unexpected cessation of motion, unforeseen and uncontrolled motion, as the current state of the art cable actuated surgical robotic end effectors all fall prey to backlash and clutching, unexpected cessation of motion, unforeseen and uncontrolled motion.

An additional exemplary embodiment is the usage of the EAP flexor extensor in combination with an elastic biasing mechanism, which may also be constructed from a highly stretchable silicone elastomer and mounted to the radiolucent joint inner housing for purposes of providing a steady pull against the rhomboidal effort arm-armature of the radiolucent joint, such that the EAP electroactive polymer musculature is set in antagonistic opposition to this biasing pull. In this manner, the elastic biasing mechanism itself acts in the manner of one of a set of flexor extensor muscles for purposes of articulation of the joint, and the EAP muscle, which is set in antagonistic opposition to this constant biasing pull, acts as the other muscle in the set of antagonist inverse proportional flexor extensors. Due to the extremely precise incremental electrical volt-by-volt pinpoint control of the expansion and compression of the EAP muscle, in opposition to the constant pull of the elastic biasing mechanism, this arrangement of EAP muscle as flexor and elastic biasing mechanism as extensor or as flexor depending upon the need exhibits an equivalent control of the articulation of all of the radiolucent joints thus presented, as the antagonist pairs of inverse proportional EAP musculature disclosed in the above. The EAP/elastic biasing mechanism pair of inverse proportional musculature does exhibit one additional advantage. Specifically, the EAP/elastic biasing flexor-extensor mechanism may remain unpowered and will relax into an articulated state. In the event of the loss of power, the elbow of the end effector would merely straighten, the wrist would merely turn, and the shoulder would relax. There is no need to for constant power. This will also facilitate transport and set up.

Other devices, apparatus, systems, methods, features, and advantages of the invention will become apparent to one with skill in the art upon examination of the following figures and detailed description. It is intended that all such additional systems, methods, features, and advantages be included within this description, within the scope of the invention, and protected by the accompanying claims.

### BRIEF DESCRIPTION OF THE DRAWINGS

The invention can be better understood by referring to the following figures. The components in the figures are not necessarily to scale; emphasis is instead placed upon illustrating the principles of the invention. In the figures, like reference numerals designate corresponding parts throughout the different views.

FIG. 1 is diagrams of a radiolucent surgical circumduction Micra-trac end-effector assembly and associated EAP muscles in accordance with an example implementation of the invention.

FIG. 2 is diagrams of the radiolucent end-effector of FIG. 1 depicting the various abduction/adduction, flexion/extension, rotation, circumduction, and one central pivot point via electroactive polymer artificial muscle and graphene cotton thread wires in accordance with an example implementation of the invention.

FIG. 3 is diagrams of the radiolucent surgical end-effector wrist of FIG. 1 depicting the various parts that make up the end-effector in accordance with an example implementation of the invention.

FIG. 4 is diagrams of a Micra-Trac radiolucent flexion/extension elbow joint utilizing EAP muscles in accordance with an example implementation of the invention.

FIG. 5 is diagrams of the Micra-Trac elbow, wrist, and end-effector pincer utilizing EAP muscles in accordance with an example implementation of the invention.

FIG. 6 is diagrams of the EAP end-effector of FIG. 1 used within a CT/MRI by a surgeon utilizing gestural gloves and a podium in accordance with an example implementation of the invention.

FIG. 7 is diagrams of a Micra-Trac radiolucent circumduction shoulder joint utilizing EAP muscle in accordance with an example implementation of the invention.

FIG. 8 is diagrams of the Micra-Trac shoulder, elbow, wrist, and end effector pincer, utilizing the Micar-Arm EAP muscle in accordance with an example implementation of the invention.

FIG. 9 is a depiction of the operation of the Micra-Arm shoulder, elbow, wrist, and pincer end-effectors actuated via EAP musculature, being used within the CT/MRI by a surgeon utilizing the gestural gloves and podium in accordance with an example implementation of the invention.

FIG. 10 is diagrams of a Micra-Arm joint with an EAP musculature and biasing member in accordance with an example implementation of the invention.

FIG. 11 is diagrams of a wrist joint using an EAP muscle bundles and biasing members of FIG. 10 in accordance with an example implementation of the invention.

FIG. 12 is diagrams of a robotic arm with multiple joints controlled by EAP muscles and biasing members in accordance with an example implementation of the invention.

### DETAILED DESCRIPTION

An embodiment is described for a non-metallic transmission of voltage to the electroactive polymer artificial muscle via the utilization of a radiolucent non-metallic flexible graphene-cotton (or other fabric) thread-based or non-metallic flexible silicone-carbon nanotube electrical transmission array for purposes of delivering voltage to and powering the antagonistic pairs (antagonistic inverse proportional pairs) of EAP flexor extensor artificial musculature. The teachings of the embodiment may also be employed in a non-radiolucent surgical robot to address the problems of backlash, clutching, unexpected cessation of motion, and unexpected and uncontrolled motion.

For purposes of attachment of these antagonistic inverse proportional pairs of EAP flexor extensors within the housing of the end effector of a surgical robot and within the radiolucent joints themselves, flexible elastomer films may be employed, as these flexible films exhibit high stretch recovery and remain permanently elastic such that each set of EAP flexor extensors may be enclosed within the joint housing in a manner which adheres the EAP musculature flat against the housing when the EAP is subjected to maximum compression and exhibits a flattened appearance, yet nonetheless expands with and continues to adhere the EAP muscle to the inner housing as the voltage decreases. The EAP muscle expands and takes on the appearance of a cube. In this manner, the flexible elastomer film acts in the manner of a balloon-like sheath, similar to the way that a human being's skin encloses the muscle of a human bicep, such that the EAP muscle always remains in the proper orientation within the inner housing of the radiolucent joints, and maintains the appropriate orientation and remains adhered throughout the entire spectrum of compression and expansion of the EAP muscle. The EAP muscles may be anchored beneath the flexible elastomer film. The EAP muscles remain correctly placed from the compressed and flattened modality through full expansion and the full spectrum of compression and expansion.

In the following exemplary embodiments, these antagonistic pairs of EAP electroactive polymer flexor extensor muscles may be utilized as one pair of antagonistic inverse proportional flexor extensors per each degree of freedom.

Example one, the one degrees of freedom radiolucent elbow joint of the present invention micra-arm may employ one pair of EAP electroactive polymer flexor extensor muscles to rotate the radiolucent rhomboidal effort arm as it performs flexion/extension.

Example two, the two degrees of freedom radiolucent circumduction joints with one central pivot point of the present invention micra-arm, wrist, and shoulder joint, may employ two pairs of EAP electroactive artificial muscle sets simultaneously. The first pair of antagonistic inverse proportional EAP artificial muscles may be used to actuate the abduction/adduction articulation function, while the second pair of antagonistic inverse proportional EAP artificial muscles may be used to actuate the flexion/extension articulating function. The circumduction function is utilized simultaneously with the first pair (abduction/adduction) and the second pair (flexion/extension).

Example three, the one-degree-of-freedom rotation joints of the upper arm/bicep and the lower arm/forearm may be employed to actuate the side-to-side rotation.

Example four, the one degrees of freedom pincer grip/scissor action of the end effector may be employed with merely one EAP artificial muscle engaged in concert with an elastic biasing mechanism, arranged such that the biasing mechanism applies a constant closing force which may be overcome and counteracted via the controlled expansion of the EAP artificial muscle, and thereby enabling the articulation motion which corresponds directly to the pincer grasp and scissoring function which replicate the radiolucent circumduction end effector usage of various forceps, drivers, retractors, and clip appliers utilized within surgical robotic microsurgery.

In this manner, the Micra-Arm is able to replicate the anatomic functions of the human wrist, elbow, and shoulder, as well as the pincer grip of the thumb and forefinger or of the index finger and thumb via the controlled delivery of voltage to the non-metallic electroactive polymer artificial EAP musculature, this voltage delivered via either the non-metallic yet conductive flexible graphene cotton thread impregnated, or via the non-metallic flexible silicone-carbon nanotube electrical transmission wire array, and coordinated via microprocessor. All movements may be conveyed directly to the end-effector via optical fiducial tracking of the surgeon motions within the gestural podium and control glove.

One advantage in the usage of the Micra-Arm is the utilization of electroactive polymers in comparison to the usage of wire/cable actuation in the present state of the art for surgical robotic joint articulation is the elimination of the “backlash” problem introduced by the buildup and sudden uncontrolled release of wire/cable tension, as well as the need for the continuous “reset” and “clutching” of the present “state of the art” wire/cable actuated end effectors.

Another significant advantage of the usage of dielectric and or ionic electroactive polymers as artificial muscle is the smooth and reliable precision control enabled in the delivery and replication of commands in the radiolucent surgical end-effector as it is utilized in microsurgical approaches in combination with graphene-infused cotton or fabric thread as the non-metallic means of conduction of said electrical impulses to the electroactive polymer artificial muscles. As stated, the ability to increase and decrease the compression of the EAP muscle incrementally and volt by volt translates into a significant enhancement of precision-based actuation and control, far in excess of the present state-of-the-art wire/cable actuation of the surgical end-effector while also eliminating the backlash hysteresis and coupling problems associated with cable actuation of the surgical robotics end-effector.

These non-metallic conductive cotton thread-based graphene or graphene nanotube silicone composites are configured as wires for the transmission of electrical current to the non-metallic parallel plate dielectric elastomer actuators arrayed as EAP antagonistic pairs of flexor extensor artificial muscles. All of these said non-metallic electrical signal wires and non-metallic electrical transmission “wires” are rendered waterproof via enclosure within flexible PVC and, or non-conductive silicone tubing, all for purposes of delivering non-metallic imaging compatible and radiolucent electrical impulse and electrical current transmission array to the surgical site within the imaging environment. These said conductive thread-based graphene-impregnated transmission wires may also be configured from any other absorbent and flexible fabric-thread-based materials in addition to cotton, to include, without limitation, rayon, hemp, linen, silk, wool, bamboo textiles, and other manufactured or natural fiber thread-based fabrics as may be amenable, as the fabric thread functions merely as the absorbent and flexible substrate. In contrast, the graphene, which has been impregnated into this flexible substrate, is the actual means of non-metallic and radiolucent electrical conduction.

All of the above radiolucent, non-metallic parallel plate dielectric elastomer actuators arrayed as EAP antagonistic pairs of flexor extensor artificial muscles, one pair per degree of freedom, are rendered electrically operable within radiographic and magnetic imaging bores via the incorporation of these radiolucent non-metallic flexible graphene-cotton thread based, or non-metallic flexible silicone-carbon nanotube electrical transmission array, all of these said components may be configured in a radiolucent non-metallic medical imaging compatible manner for usage & incorporation within the radiolucent surgical robotics circumduction end effectors, to include the sheathing of the effector housings within flexible yet non-conductive silicone elastomer film all along the exterior portions, for purposes of insulating the housing and to safeguard the entire end-effector apparatus itself from transmitting current to the patient. As disclosed, the non-metallic wires may be sheathed in non-conductive tubing, the EAP artificial muscles may be encased within non-conductive elastomer film, and the entire housing of the end-effector may be placed within a non-conductive silicone sheath, thereby enabled to transit voltage to the electroactive EAP pairs of antagonistic inverse proportional flexor extensor artificial muscle, in a manner which insulates the end-effector from transmitting current to the patient.

An alternative embodiment of the Micra-Arm/Micra-Trac radiolucent end-effector with gestural haptic control glove may also be incorporated within radiolucent flexible surgical robotics platforms via positioning of the Micra-Arm EAP radiolucent circumduction end effector at the distal tip of the flexible robot, with all non-metallic cotton thread-based graphene wires and, or graphene silicone electrical conductivity wires, configured to pass through a series of central foramina magna (plural of foramen magnus) which may then be utilized to power the EPA Musculature for purposes of effecting articulation. This way, the radiolucent circumduction end-effector and flexible surgical robotic platform may be further enabled to perform surgery within previously inaccessible sites.

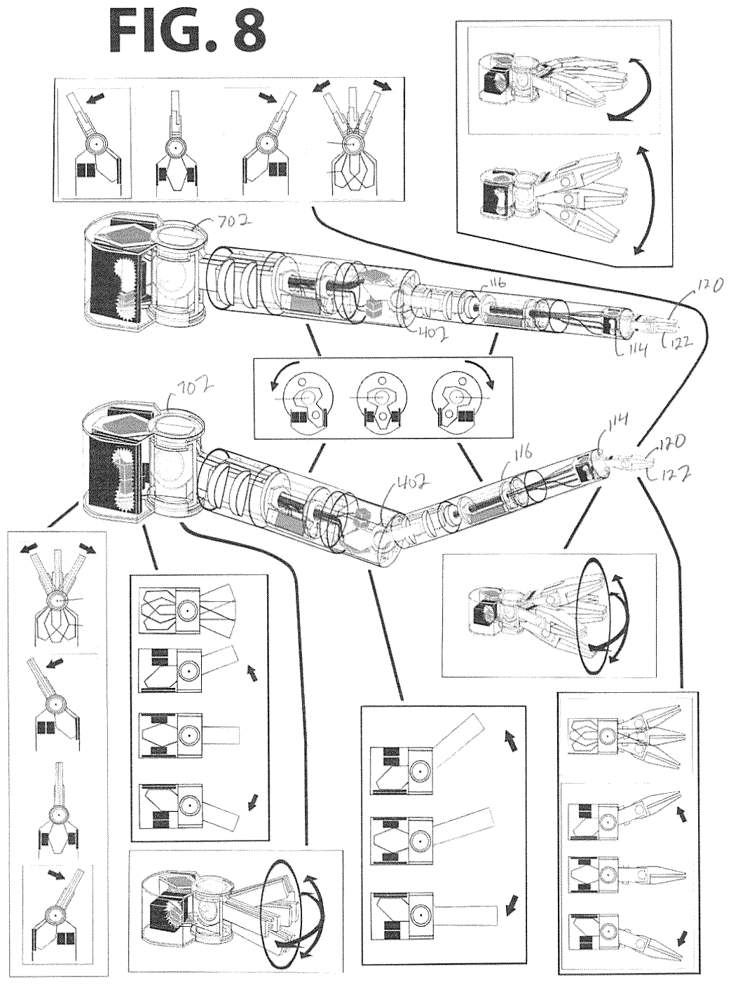

Yet another embodiment of the Micra-Arm radiolucent end-effector, such as shown in FIG. 8, is a single port iteration of the present invention configured as a radiolucent circumduction end effector with radiolucent shoulder, radiolucent elbow, and radiolucent wrist, configured via the combination of the radiolucent three degrees of freedom circumduction joint and the radiolucent one degree of freedom flexion-extension joint. This iteration of a radiolucent Micra-Reach radiolucent shoulder, elbow, and wrist (SEW) end-effector is disclosed for purposes of enabling the distal triangulation of multiple Micra-Reach SEW radiolucent end-effectors through one radiolucent uni-port cannula for single port access of minimally invasive surgical procedures. One example of such a minimally invasive medically imaging-compatible procedure would be the 3D CT/computerized tomography image-guided in-utero microsurgery to correct for fetal cardiac or Spina-Bifida malformations, with surgical access to the womb enabled via a single radiolucent port. Via this single radiolucent port, multiple of the Micra-Arm radiolucent end effectors may access the surgical site via distal triangulation, with surgeon gestural control enabled via optical fiducial makers and image capture of a sleeved garment arrayed with optical fiducial markers from the fingertips to the surgeon's shoulders, thereby allowing the surgeon to control, two of these said radiolucent Micra-Arm end effectors at once and entirely via gestural motions of the surgeon's fingers, thumbs, hand, wrist, elbow and shoulder.

In this iteration, the surgeon may remain comfortably seated within a chair with arm and shoulder rests and control the radiolucent Micra-Arm end effector via the naturalistic motions of the shoulder, elbow, wrist, fingers, and thumb. For the most part, the surgeon's gestural control will involve the elbow, wrists, fingers, and thumb, with only occasional gestural control of the shoulder. A large screen may be employed for usage with 3D glasses, such that the surgeon may sit comfortably either before this large screen while performing surgery or being equipped with a VR headset with a heads-up augmented reality display through which the surgeon may visualize not only the images captured by the fiber optics array but may also visualize the 3D image guided scan, as well as various combinations of both the optical and the image-guided scan superimposed atop one another.

To reiterate, in this manner, the surgeon is enabled to perform the most intricate micro-surgery with all of the skill of the surgeon's actual hands and digits reduced to the micro level, and yet with none of the dangerous lack of accurate control caused by the coupling phenomenon, backlash and hysteresis presented by cable actuation of surgical end effectors and with all of the benefit of the most advanced CT and MRI real-time 3D imaging guidance at their disposal. Furthermore, all of the above are thereby enabled to function within the MRI, CT, O-arm, and radiographic imaging bores in a non-metallic medically imaging compatible and radiolucent manner, which neither significantly affects the quality of the diagnostic information nor has its operations affected by the medical imaging system.

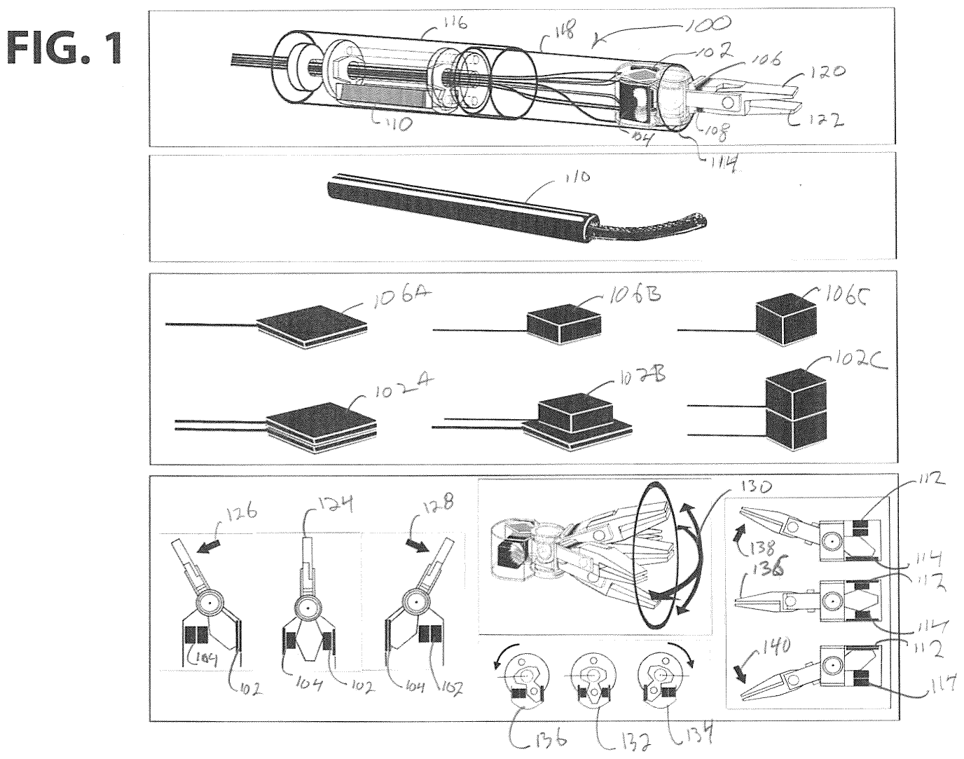

In FIG. 1, diagrams of a radiolucent surgical circumduction Micra-trac end-effector assembly 100 and associated EAP muscles 102-110 are depicted in accordance with an example implementation of the invention. The assembly 100 enables housing 116 and 118 to be slidably coupled and controlled by EAP muscle 110 with an electrical current. As an EAP muscle receives current, it contracts or flexes. This movement of the EAP muscle translates to movement of the joints, such as joint 114 and end-effector pincers 120, 122. A single EAP muscle has a predetermined rest state, such as 106A. As current is removed or reduced via a conductor, such as a graphene-impregnated thread, the EAP muscle expands 106B. The EAP muscle has a maximum expansion of 106C that can be preset by the reduction in current or via its inherent structure, depending upon the implementation.

Similarly, more than one EAP muscle may be used together 102A. Current may be passed to one EAP muscle of muscle bundle 102B and not others or to all of the EAP muscles of muscle bundle 102C. By bundling EAP muscles, an increased area of EAP muscle movement is achieved, and force is spread out among the EAP muscles in the EAP muscle bundle. For example, the EAP muscle bundle moves 124-126 in joint 114, with a rhomboidal member controlled by an inverse EAP muscle bundle pair 102 and 104. Similarly, inverse EAP muscle bundle pairs control the wrist joint movement 130-140.

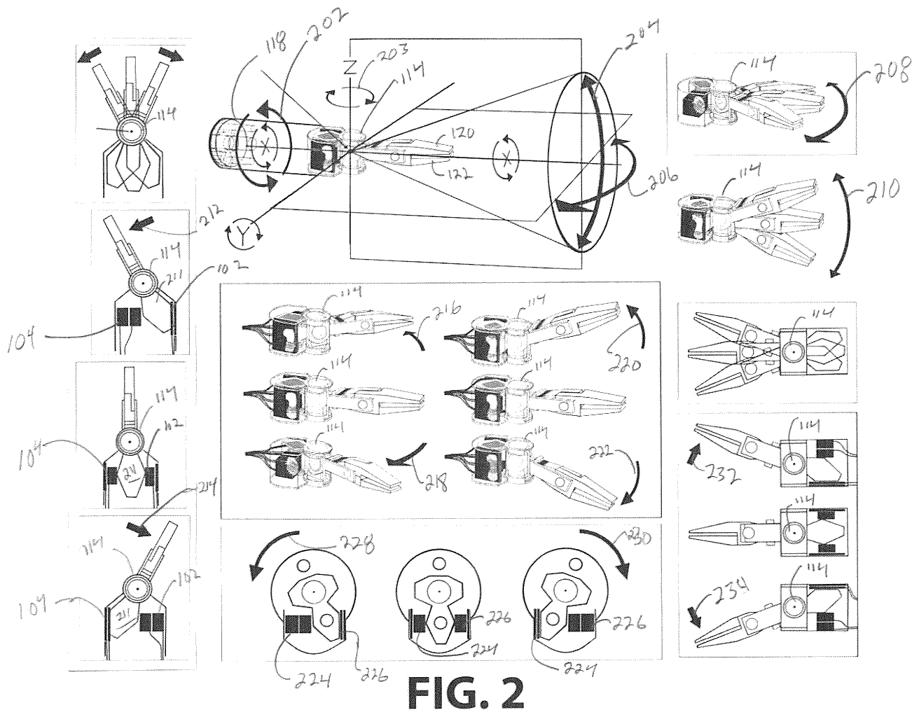

Turning to FIG. 2, diagrams of radiolucent end-effector 114 of FIG. 1 depicting the various abduction/adduction, flexion/extension, rotation, circumduction, and one central pivot point via electroactive polymer artificial muscle, and graphene cotton thread wires is shown in accordance with an example implementation of the invention. The end-effector's pincers 120, 122 and wrist joint 114 can rotate in the x-axis 202 along with the pincers 120, 122. The wrist joint 114 can rotate in the z-axis 203, resulting in the pincers 120 122 also moving in the z-axis 206. The pincers 120 122 can also move in the y-axis 204. A closer-up view of the EAP muscles movement of the pincers at the wrist joint is shown in 208 and 210. An overhead view of the wrist joint 114 moving in the z-axis is demonstrated 212, 214, where the inverse EAP muscle pair 102 and 104 act on a rhomboidal member 211 to facilitate the movement.

Similarly, the wrist joint 114 movements are accomplished in the z-axis and y-axis 216-22. The rotation of the wrist joint in the X-axis 228 and 230 is performed with an EAP muscle pair 224 and 226 acting on a rhomboidal member. The y-axis movement of the wrist is further shown in 232 and 234.

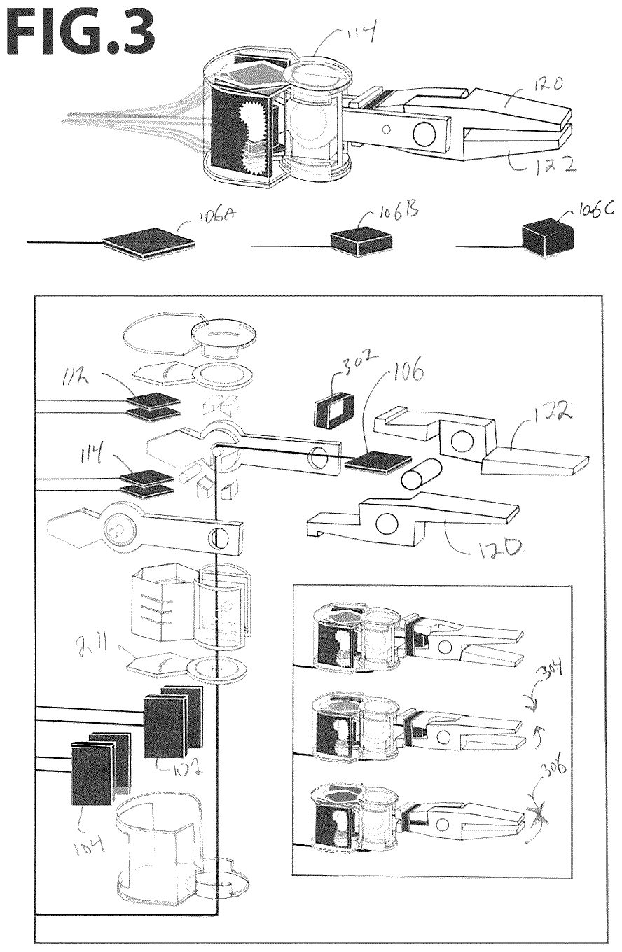

In FIG. 3, diagrams of the radiolucent surgical end-effector wrist 114 of FIG. 1 depicting the various parts that make up the end-effector wrist 114 is shown in accordance with an example implementation of the invention. The components that make up the wrist are shown with an example of a single EAP muscle in different states of activation 106A-106B used to open the pincers 120, 122, that biased in the closed position 304, 306 by band 302. The EAP muscles are coupled to power sources via non-metallic graphene-impregnated cotton thread wires. The inverse EAP muscle pairs are depicted 112 and 114, 102 and 104, and act on rhomboidal members, such as 102 and 104 acting on rhomboidal member 211.

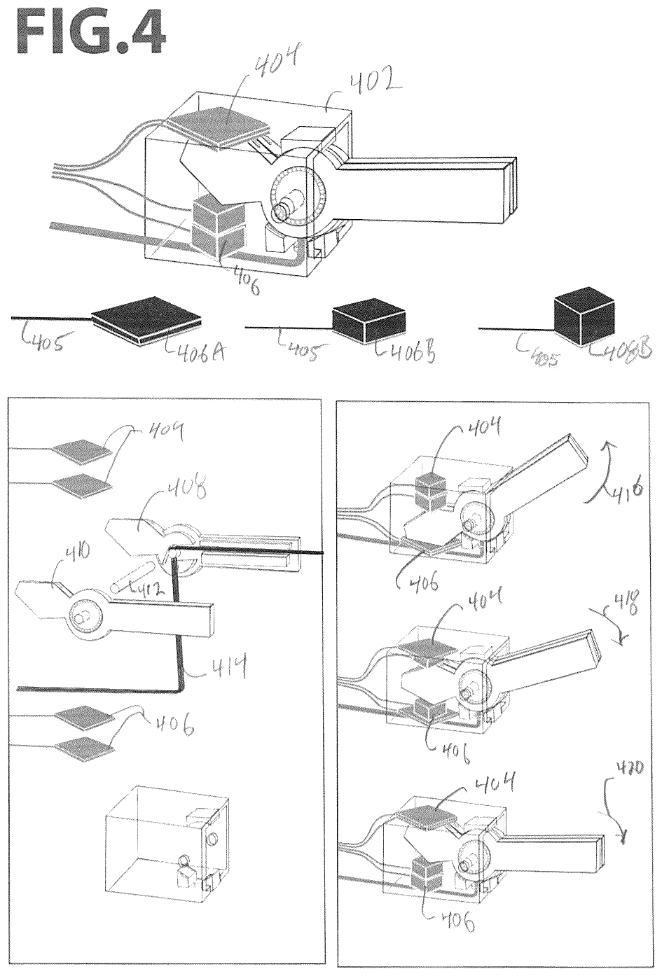

Turning to FIG. 4, diagrams of a Micra-Trac radiolucent flexion/extension elbow joint 402 utilizing EAP muscles 404, 406 in accordance with an example implementation of the invention. An example of one of the EAP muscles being powered via the reduction of current along non-metallic graphene-impregnated cotton thread wire 405. A pair of rhomboidal members 408 and 410 are coupled by axel 412 with a bundle of non-metallic graphene-impregnated cotton thread wire 414 that are routed to other EAP muscles (not shown). The inverse EAP muscle pair 404 406 act upon the rhomboidal members. When EPA muscle bundle 404 is activated, and inversely bundle 406 is deactivated (full current), the elbow flexes up 416. When EPA muscle bundle 404 is deactivating while EAP muscle bundle is activating 406, the elbow flexes downward 418. The elbow joint is fully extended when EAP muscle bundle 404 is completely deactivated, and EAP muscle bundle 406 is activated.

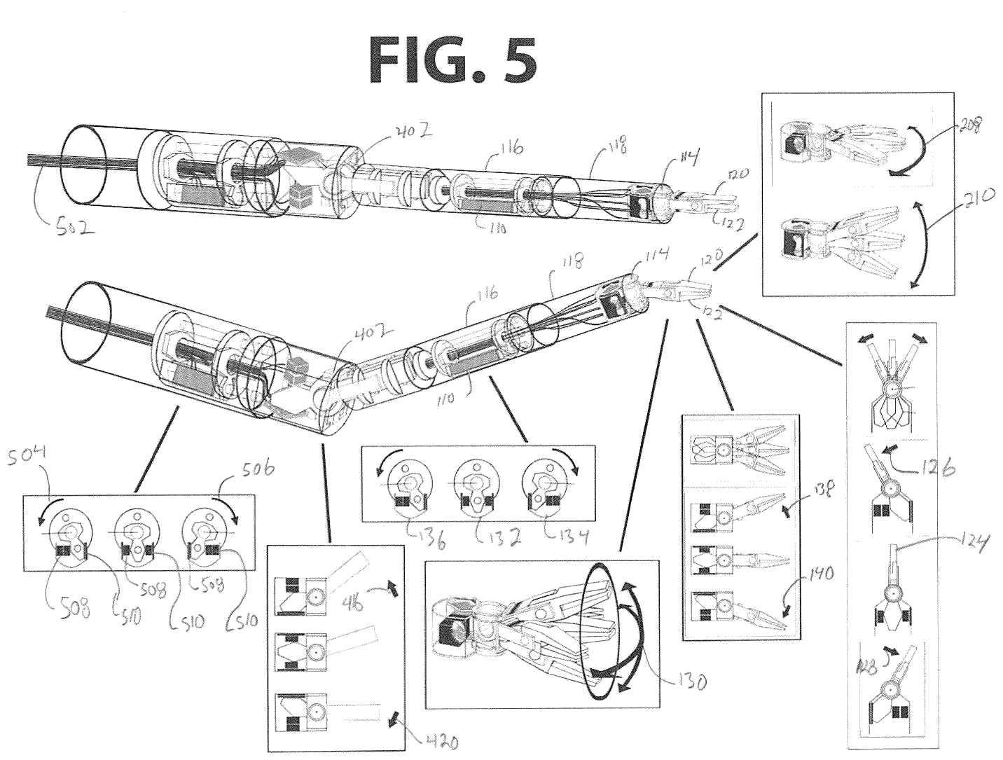

In FIG. 5, diagrams of the Micra-Trac elbow 402, wrist 114, and end-effector pincer 120, 122 utilizing EAP muscles are shown in accordance with an example implementation of the invention. All articulations and EAP muscles are depicted, with the specific articulation functions of rotation, flexion/extension, circumduction, abduction/adduction all represented and displaying the compression and expansion of the EAP musculature. A plurality of non-metallic graphene impregnated cotton thread wires 505 are passed through the center of the arm and are coupled to the plurality of EAP muscles. Rotation 504 and 506 of the elbow joint 402 is accomplished by an inverse EAP muscle par 508 and 510 acting on a rhomboidal member. Other previous described movements are reiterated in FIG. 5.

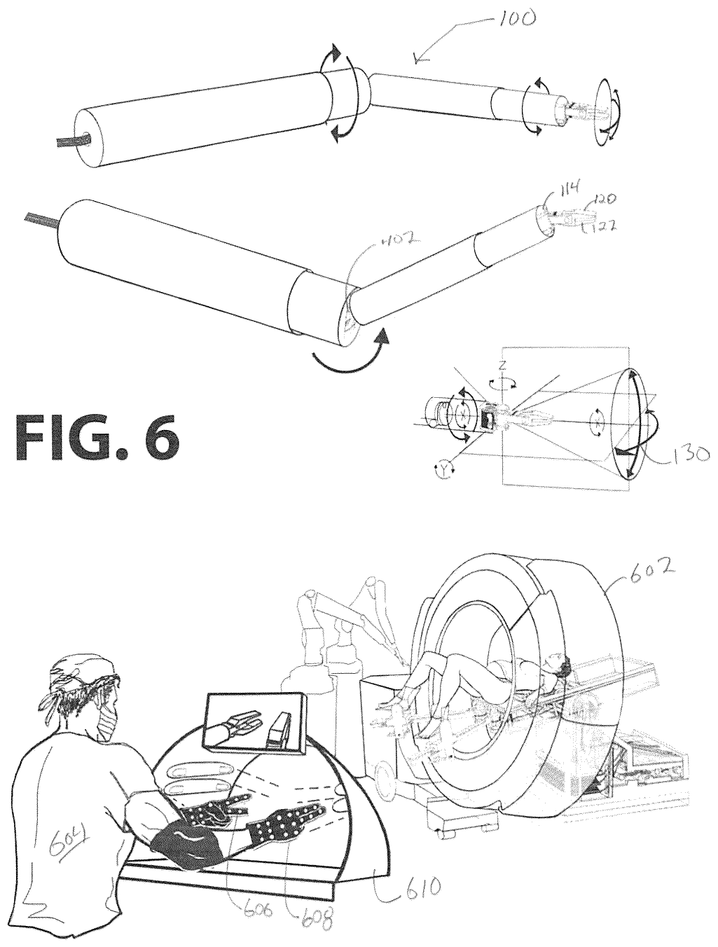

Turning to FIG. 6, diagrams of the EAP end-effector 100 of FIG. 1 used within a CT/MRI 602 by a surgeon 604 utilizing gestural gloves 606 and 608 and podium 610 is shown in accordance with an example implementation of the invention.

An alternative embodiment of the Micra-Arm/Micra-Trac radiolucent end-effector with gestural haptic control glove 606, 608 may also be incorporated within radiolucent flexible surgical robotics platforms via positioning of the radiolucent circumduction end effector at the distal tip of the flexible robot, with all non-metallic cotton thread-based graphene wires and, or graphene silicone electrical conductivity wires, non-metallic cable actuation wires, and/or pneumatic lines configured to pass through a series of central foramina magna (plural of foramen magnus) which may then be utilized to power the Electroactive Polymer Artificial Musculature for purposes of effecting articulation. This way, the radiolucent circumduction end-effector and flexible surgical robotic platform may be further enabled to perform surgery within previously inaccessible sites.

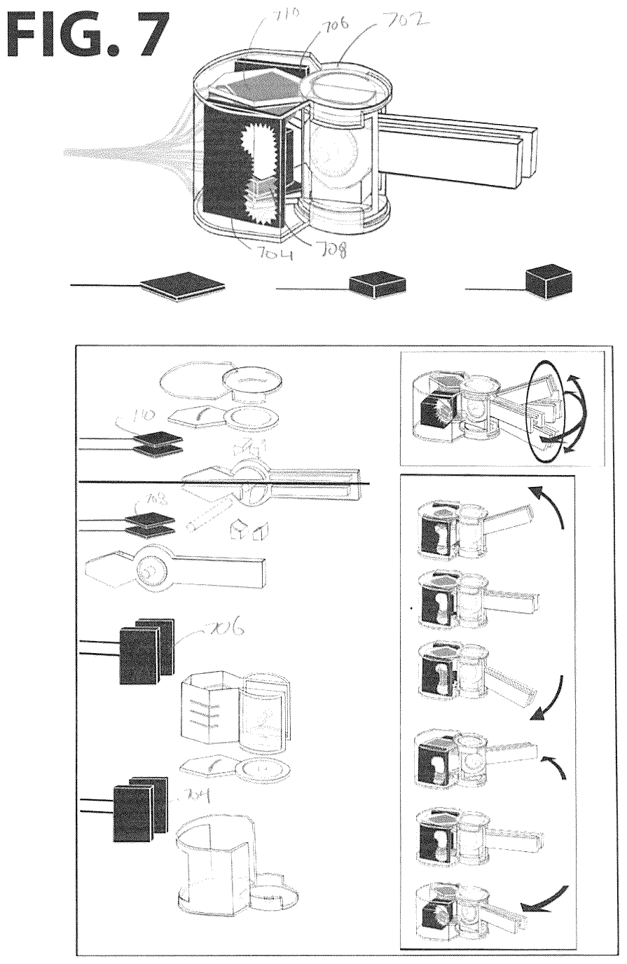

In FIG. 7, diagrams of a Micra-Trac radiolucent circumduction shoulder joint 702 utilizing inverse EAP muscle pairs 704, 706, 708, and 710 are presented in accordance with an example of the implementation of the invention. The parts of the shoulder joint 702 are similar to the parts and function 114 of FIG. 3.

Turning to FIG. 8, diagrams of the Micra-Trac shoulder 702, elbow 402, wrist 114, and end-effector pincer 120, 122, utilizing the Micar-Arm EAP muscles, is shown in accordance with an example implementation of the invention. Yet another embodiment of the Micra-Arm radiolucent end-effector with FIG. 8, is a single port iteration of the present invention configured as a radiolucent circumduction end effector with radiolucent shoulder, radiolucent elbow, and radiolucent wrist, configured via the combination of the radiolucent three degrees of freedom circumduction joint and the radiolucent one degree of freedom flexion-extension joint.

This embodiment of a radiolucent Micra-Reach radiolucent shoulder, elbow, and wrist (SEW) end-effector (Micra-Reach SEW radiolucent end-effector) is disclosed for purposes of enabling the distal triangulation of multiple Micra-Reach SEW radiolucent end-effectors through one radiolucent uni-port cannula for single port access of minimally invasive surgical procedures. One example of such a minimally invasive medically imaging-compatible procedure would be the 3D CT/computerized tomography image-guided in-utero microsurgery to correct for fetal cardiac or Spina-Bifida Malformations, with surgical access to the womb enabled via a single radiolucent port. Via this single radiolucent port, multiple of the Micra-Arm radiolucent end effectors may access the surgical site via distal triangulation, with surgeon gestural control enabled via optical fiducial makers and image capture of a sleeved garment arrayed with optical fiducial markers from the fingertips to the surgeon's shoulders, thereby allowing the surgeon to to control, two of these said radiolucent Micra-Arm end effectors at once and entirely via gestural motions of the surgeon's fingers, thumbs, hand, wrist, elbow and shoulder. In this iteration, the surgeon may remain comfortably seated within a chair with arm and shoulder rests and control the radiolucent Micra-Arm end effector via the naturalistic motions of the shoulder, elbow, wrist, fingers, and thumb, wrists, fingers, and thumb, with only occasional gestural control of the shoulder. To reiterate, in this manner, the surgeon is enabled to perform the most intricate micro-surgery with all of the skill of the surgeon's actual hands and digits reduced to the micro level, and yet with none of the dangerous lack of accurate control caused by the coupling phenomenon, backlash and hysteresis presented by cable actuation of surgical end effectors and with all of the benefit of the most advanced CT and MRI real-time 3D imaging guidance at their disposal.

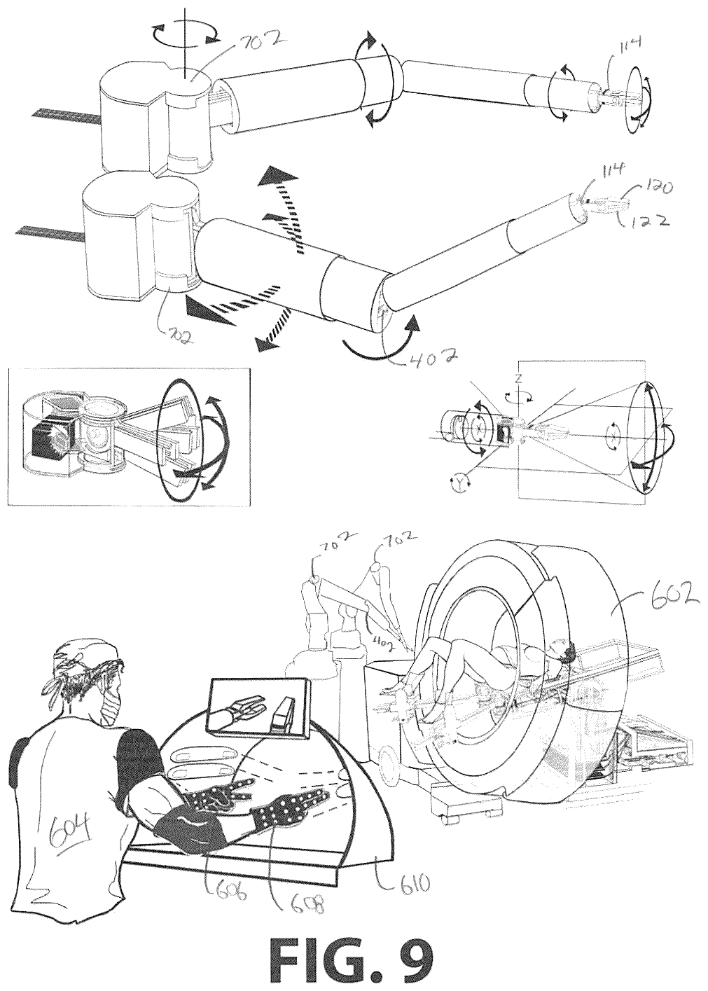

In FIG. 9 depictions of the operation of the Micra-Arm shoulder 708, elbow 402, wrist 114, and pincer end-effectors 120, 122, actuated via EAP musculature, being used within the CT/MRI 602 by a surgeon 604 utilizing the gestural gloves 606, 608 and podium 610 is shown in accordance with an example implementation of the invention.

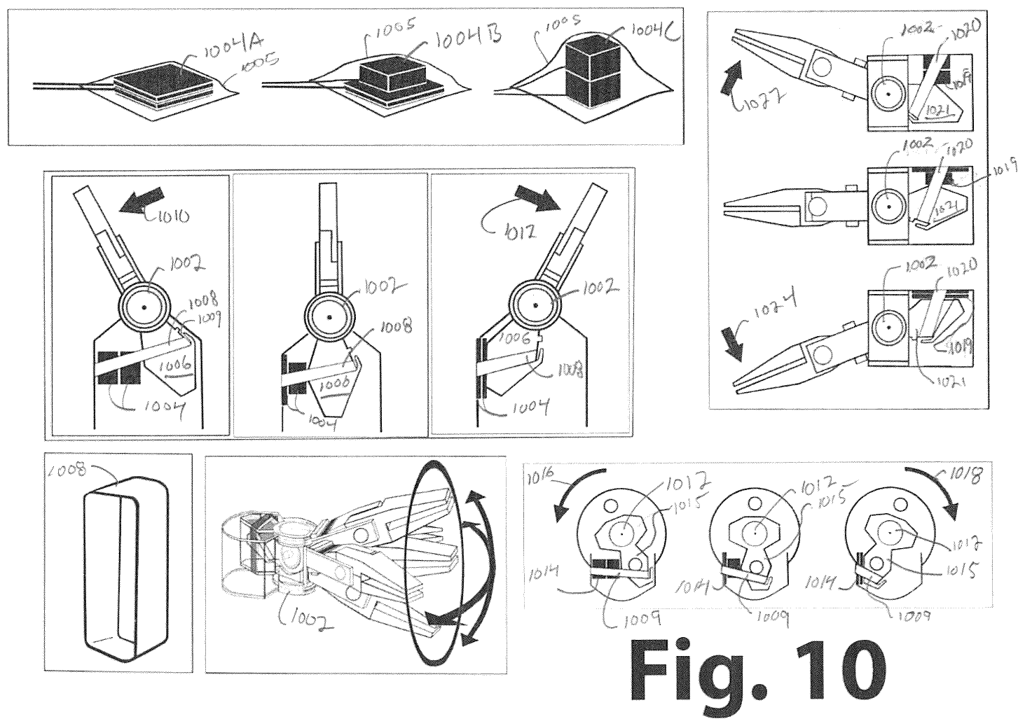

Turning to FIG. 10, diagrams of a Micra-Arm joint with an EAP musculature and biasing member in accordance with an example implementation of the invention. The EAP muscle is depicted as two EAP muscles 1004 with associated leads for supplying current with the EAP muscles 1004 is draped beneath Highly Stretchable Sheets of Silicone Elastomer (HSSSE) 1005. In an energized or compressed state, that EAP muscle uses the least amount of area 1004A, with the HSSSE 1005 applying the least amount of force to the EAP bundle 1004. With two or more EAP muscles working in an EAP muscle bundle, each may be activated individually 1004 or both de-energized at the same time 1004C with an associated increase in force applied by the HSSSE 1005 to the EAP muscle bundle 1004B and 1004C. A Micra-Arm joint 1002 may be structured with a single EAP muscle 1004 and a biasing member 1008. The EAP muscle 1004 provides a force against a rhomboidal member 1006 in opposition to the biasing force applied to the rhomboidal member 1006 by the biasing member 1008.

The purpose of the HSSSE 1005 is to enable the EAP muscles to remain in the proper orientation, such that when fully compressed and flat, the HSSSE 1005, which is attached to the radiolucent joint inner housing, remains snug against the flattened EAP muscles 1004A. Correspondingly, as the EAP muscles undergo expansion due to the variable removal of current 1004B and even become cube-like when subjected to no current and in a state of maximum expansion, the HSSSE remains snug against the EAP musculature and withholds these EAP muscles in the exact proper orientation.

The usage of a biasing member 1008, an elastic biasing mechanism in the current embodiment, which may also be constructed from a highly stretchable silicone elastomer and mounted to the radiolucent joint inner housing for purposes of providing a steady pull against the rhomboidal effort armature 1006 of the radiolucent joint, such that the EAP musculature 1004 is set in antagonistic opposition to this biasing pull. A notched portion 1009 of the rhomboidal effort arm for purposes of attachment of the elastic biasing mechanism is depicted in the current embodiment. In this manner, the elastic biasing mechanism itself acts in the manner of one of a set of flexor extensor muscles for purposes of articulation of the joint 1002, and the EAP muscle bundle 1004, which is set in antagonistic opposition to this constant biasing pull acts as the other muscle in the set of antagonists inverse proportional flexor extensors. Due to the exact incremental electrical current application, pinpoint control of the expansion and compression of the EAP muscle bundle 1004 is achieved, in opposition to the constant pull of the elastic biasing mechanism 1008, this arrangement of EAP muscle bundle 1004 as a flexor. An elastic biasing mechanism, such as an extensor (or vice versa, depending on the need), exhibits an equivalent control of the articulation of all of the radiolucent joints thus presented.

The movement of the joint 1010, 1012, 1016, 1018, 1022, 1024 of the joints 1002, 1012, and 1002 is achieved by using an EAP muscle bundle 1004, 1014, 1019 acting on a rhomboidal member 1006 1015, 1021 against the force of biasing member 1008, 1009, 1020.

The EAP/elastic biasing mechanism pair of inverse proportional musculature does exhibit one additional advantage. Specifically, the EAP/elastic biasing flexor-extensor mechanism may remain unpowered and will relax into an articulated state. In the event of the loss of power, the elbow of the end effector would merely straighten, the wrist would merely turn, and the shoulder would relax. There is no need for constant power. This will also facilitate transport and set up. In other implementations, dual-equal biasing may be employed to maintain the joint in a center position when power is not available, and inverse EAP muscle bundle pairs are used to act against the biasing to move the joint from a steady state center position.

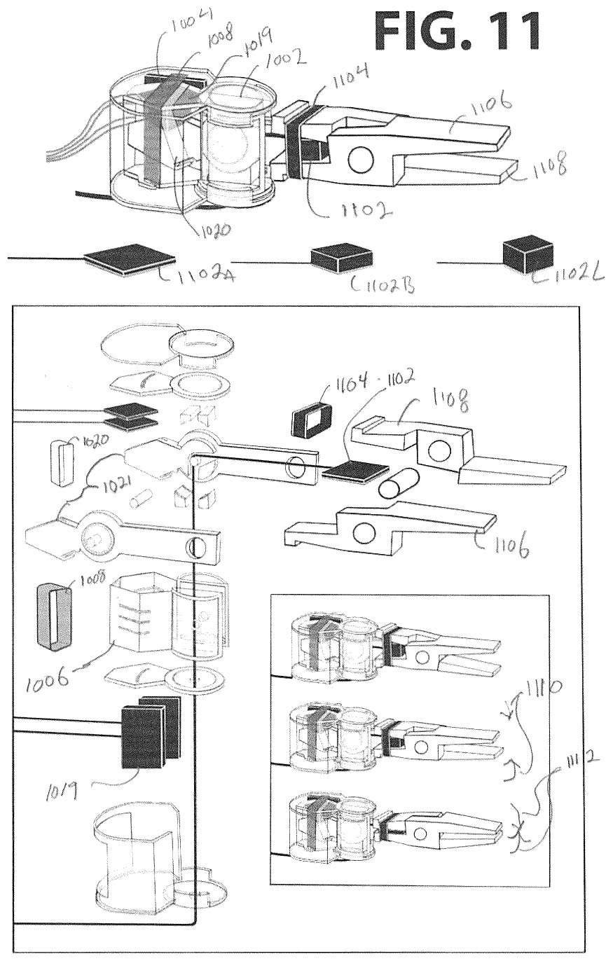

In FIG. 11, diagrams of a wrist joint 1002 using EAP muscles 1004, 1019, 1102 and biasing members 1008, 1020, and 1104 of FIG. 10 are shown in accordance with an example implementation of the invention.

The elastic biasing mechanism utilized for flexion-extension is shown in white, and the elastic biasing mechanism utilized for abduction/adduction is shown in grey with the EAP muscle 1002 depicted in a powered 1102A, 1102B and compressed (unpowered) 1102C state. The elastic biasing mechanisms are mounted to the inner housing of the radiolucent joint in order to exhibit a constant force in opposition to the expansion of the electroactive polymer muscle, in the manner of an extensor in opposition to a flexor or vice-versa depending on the intended usage of the articulation as needed.

The top illustration depicts the internal flexion-extension elastic biasing mechanism as constant force/pressure is applied in opposition to the EAL muscle bundle. This flexion-extension EAP/elastic biasing mechanism pair of flexor extensors is shown in white as the band 1020 inside of the rotating abduction/adduction module, upon which may be seen the grey elastic biasing mechanism 1008 being utilized as an extensor, against which the EAP muscles is in constant opposition. The opening and closing 1110, 1112 of the radiolucent pincer 1106, 1008 via this same EAP 1102 and biasing mechanism 1104 acting in opposition.

The EAP muscle/biasing mechanism as an antagonistic flexor extensor may also be employed to affect the articulation of flexible robotics and radiolucent non-metallic flexible robotics in other embodiments. In yet other embodiments, a combination of EAP muscle, EAP muscle/biasing, cable, and pneumatic bags may be used together. The EAP muscle and/or EAP muscle/biasing mechanism may be employed in an articulation of flexible robotics like robotic art.

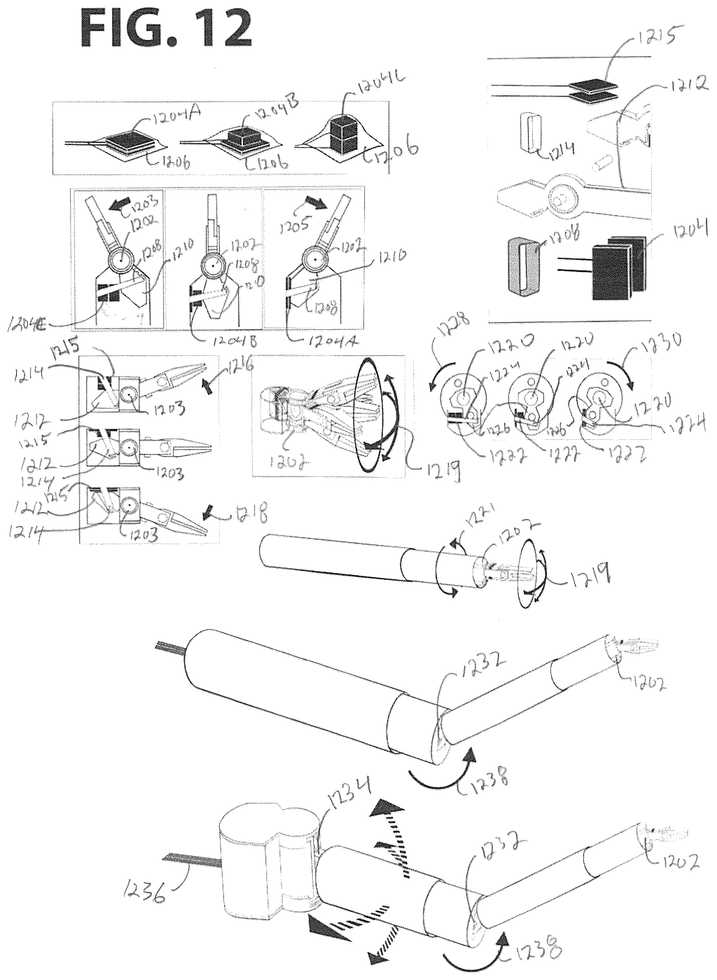

Turning to FIG. 12, diagrams of a robotic arm with multiple joints 1234, 1232, and 1202 controlled by EAP muscles 1204, 1215, 1222 and biasing members 1208, 1214, and 1224 are shown in accordance with an example implementation of the invention. The muscle bundles, such as 1204, are enclosed in HSSSE 1206. When fully activated, the HSSSE 1206 aids in the muscle bundle to maintain its orientation 1204. As the current is reduced, the HSSSE 1206 is stretched 1204B until the current is completely removed or in its lowest state, 1204C.

When the EAP muscle bundle is in its lowest power state, 1204C, the joint 1202 is moved to a first position 1203 and act against the biasing member 1208. As current is applied to the EAP muscle bundle 1204B, the joint moves, and when fully powered 1204C, the joint is moved to a biased position 1205. Similar movements 1216, 1218, 1219, 1221, and 1238 are similarly accomplished with joints 1203, 1232, and 1234 (for abduction/adduction. Flexion/extension, circumduction, and rotation) having EAP muscles 1215, 1222 acting against rhomboidal members 1210, 1212, 1226 that biased by biasing members 1214, 1224. The EAP muscles are coupled to graphene-impregnated cotton thread 1236 that acts as a conduit for current from an associated controller that can interface to other input and output devices.

Micra-reach enhances the surgeon's experience via the addition of carbon-based non-metallic and silicone elastomer EAP Artificial Muscles Non-metallic conductive cotton thread-based graphene for transmission of electric current to the non-metallic bipolar electrocautery and for the transmission of signals from the piezo-electric strain gauges and sensors arrayed in the end effectors to the surgeon's gestural control glove is depicted in the current embodiment. Furthermore, the usage of radiolucent, medically imaging compatible, non-metallic conductive graphene thread and or Silicone Graphene Nanotubes as the means of electrical current transmission means of electrical current transmission may be enclosed within flexible and radiolucent PVC and or silicone tubing for purposes of waterproofing and thereby rendered accessible and fully operable within the surgical site.

It will be understood and appreciated by persons skilled in the art that one or more processes, sub-processes, or process steps may be performed by hardware and/or software (machine-readable instructions). If the approach is performed by software, the software may reside in software memory in a suitable electronic processing component or system, such as one or more of the functional components or modules schematically depicted in the figures.

The software in software memory may include an ordered listing of executable instructions for implementing logical functions (that is, “logic” that may be implemented either in digital form, such as digital circuitry or source code, or in analog form, such as analog circuitry or an analog source such an analog electrical, sound or video signal), and may selectively be embodied in any computer-readable medium for use by or in connection with an instruction execution system, apparatus, or device, such as a computer-based system, processor-containing system, or other systems that may selectively fetch the instructions from the instruction execution system, apparatus, or device and execute the instructions. In the context of this disclosure, a “computer-readable medium” is any tangible means that may contain or store the program for use by or in connection with the instruction execution system, apparatus, or device. The tangible computer-readable medium may selectively be, for example, but is not limited to, an electronic, magnetic, optical, electromagnetic, or semiconductor system, apparatus, or device. More specific examples, but nonetheless a non-exhaustive list, of tangible computer-readable media would include the following: a portable computer diskette (magnetic), a RAM (electronic), a read-only memory “ROM” (electronic), an erasable programmable read-only memory (EPROM or Flash memory) (electronic) and a portable compact disc read-only memory “CDROM” (optical). Note that the tangible computer-readable medium may even be paper (punch cards or punch tape) or another suitable medium upon which the instructions may be electronically captured, then compiled, interpreted, or otherwise processed in a suitable manner if necessary, and stored in computer memory.

The foregoing detailed description of one or more embodiments has been presented herein by way of example only and not limitation. It will be recognized that there are advantages to certain individual features and functions described herein that may be obtained without incorporating other features and functions described herein. Moreover, it will be recognized that various alternatives, modifications, variations, or improvements of the above-disclosed embodiments and other features and functions, or alternatives thereof, may be desirably combined into many other different embodiments, systems, or applications. Presently unforeseen or unanticipated alternatives, modifications, variations, or improvements therein may be subsequently made by those skilled in the art, which are also intended to be encompassed by the appended claims. Therefore, the spirit and scope of any appended claims should not be limited to the description of the embodiments contained herein.

What is claimed is:

1. A robot, comprising:
a robotic base;
an at least one end-effector coupled to the base with an at least one joint;
an at least one ElectroActive Polymer (EAP) muscle that positions the at least one joint and engages with a movable joint member;
a biasing member that provides a biasing force acting against the force provided by the EAP muscle.

2. The robot of claim 1, where the EAP muscle is coupled to a current source via a wire.

3. The robot of claim 2, where the wire is a graphene-impregnated thread.

4. The robot of claim 2, where the wire is graphene nanotube silicone wire.

5. The robot of claim 1, where the at least one joint, at least one end effector, EAP muscle, and biasing member are radiolucent when in an MRI/CT bore.

6. The robot of claim 1, where the biasing member is a second EAP muscle that provides an inverse force to the at least one EAP muscle.

7. The robot of claim 1, where the biasing member is a stretchable biasing member.

8. The robot of claim 7, where the stretchable biasing member is Silicone Elastomer.

9. The robot of claim 1, where the EAP muscle is constrained and maintains its position in the joint no matter the state of the EAP muscle.

10. The robot of claim 9, where the EAP muscle is constrained by being encased in a Silicone Elastomer bag.

11. The robot of claim 9, where the movable joint member has at least a portion that is rhomboidal shaped and engaged by the EAP muscle.

12. The robot of claim 11, where the biasing member engages with the at least a portion of the movable joint member.

13. A method for moving a robotic joint, comprising:
coupling a joint in an at least one end-effector to a base;
moving the joint with at least one EAP muscle engaging a joint member; and
opposing the movement of the at least one EAP muscle with a biasing member.

14. The method of claim 13, includes a current source, where the EAP muscle is coupled to the current source via a wire.

15. The method of claim 13, where the wire is a graphene-impregnated thread.

16. The method of claim 13, where the at least one joint, at least one end effector, EAP muscle, and biasing member are radiolucent when in an MRI/CT bore.

17. The method of claim 13, where biasing with the biasing member further includes, a second EAP muscle that provides an inverse force to the at least one EAP muscle.

18. The method of claim 17, where the stretchable biasing member is Silicone Elastomer.

19. The method of claim 13, includes constraining the EAP muscle to maintain its position in the joint no matter the state of the EAP muscle.

20. The method of claim 19, where the EAP muscle is constrained by being encased in a Silicone Elastomer bag.

21. The method of claim 13, where the joint member has at least a portion that is rhomboidal shaped.
